(19) United States
(12) Patent Application Publication
(10) Pub. No.: US 2018/0214033 A1
(43) Pub. Date: Aug. 2, 2018

(54) HEMODYNAMIC MONITOR PROVIDING ENHANCED CARDIAC OUTPUT MEASUREMENTS

(71) Applicant: Edwards Lifesciences Corporation, Irvine, CA (US)

(72) Inventor: Alexander Holland, Santa Ana, CA (US)

(73) Assignee: Edwards Lifesciences Corporation, Irvine, CA (US)

(21) Appl. No.: 15/885,232

(22) Filed: Jan. 31, 2018

Related U.S. Application Data
(60) Provisional application No. 62/453,754, filed on Feb. 2, 2017.

(51) Int. Cl. A61B 5/02; A61B 5/029; A61B 5/0215

(52) U.S. Cl. CPC A61B 5/02028; A61B 5/0004; A61B 5/02156; A61B 5/029

(57) ABSTRACT

A hemodynamic monitor implements an adaptive method that optimally estimates scaling and offset calibration parameters by using a computationally efficient, iterative online method to minimize the mean square error between a high bandwidth arterial pressure cardiac output (APCO) measurement generated by a first physiological sensor affixed to a patient and a relatively low bandwidth continuous cardiac output (CCO) measurement generated by a second physiological sensor also affixed to the patient. When calibration parameters are used to adjust an APCO measurement, the combined APCO/CCO estimate provided by the hemodynamic monitor has accuracy comparable to a CCO measurement, but also tracks cardiac output dynamical variations that are outside of the CCO algorithm bandwidth.

### RELATED APPLICATION

This application claims priority to U.S. Pat. App. Ser. No. 62/453,754 filed on Feb. 2, 2017, the contents of which are hereby fully incorporated by reference.

### TECHNICAL FIELD

The subject matter described herein relates to hemodynamic monitors utilizing two or more different physiological sensors to provide enhanced cardiac output measurements.

### BACKGROUND

Cardiac output (CO) provides an indication of the volume of blood being pumped by the heart of a patient at any given time. There are numerous techniques for determining cardiac output, including the arterial pressure cardiac output (APCO) algorithm, and the continuous cardiac output (CCO) and injectate cardiac output (ICO) thermal dilution style algorithms. APCO style algorithms generally have significantly higher bandwidth than CCO or ICO algorithms. Given such an arrangement, APCO algorithm measurements are less averaged or collected more regularly, and hence can better follow rapid or transitory changes in patient cardiac output CO. However, APCO algorithms with peripheral pressure signals as input are in general less accurate than CCO or ICO algorithms that directly measure central blood flow.

### SUMMARY

In one aspect, first data is continuously received that is generated by a first physiological sensor measuring at least one hemodynamic parameter of a patient. In addition, second data is continuously received that is generated by a second physiological sensor concurrently measuring the at least one hemodynamic parameter of the patient. The first physiological sensor measures the at least one hemodynamic parameter at a higher bandwidth with lower precision as compared to the second physiological sensor. The continuously received first data is adaptively calibrated using the continuously received data to result in a continually updating calibrated measurement. Data characterizing the continually updating calibrated measurement can then be provided.

The providing data can take many forms including, for example, one or more of: displaying the data characterizing the calibrated measurement in an electronic visual display, transmitting the data characterizing the calibrated measurement to a remote computing system, loading the data characterizing the calibrated measurement into memory, and/or storing the data characterizing the calibrated measurement in physical data persistence.

The at least one hemodynamic parameter can be cardiac output.

The first physiological sensor can be used to measure arterial pressure cardiac output. The first physiological sensor can include a cuff to be placed on an extremity of the patient and utilizing a volume clamp method to calculate one or more of: stroke volume, stroke volume variation, APCO, systemic vascular resistance (SVR), and/or continuous blood pressure (cBP).

The second physiological sensor can be used to measure continuous cardiac output and/or injectate cardiac output. The second physiological sensor can include a pulmonary artery catheter (PAC) that is inserted into a pulmonary artery of the patient to detect cardiac pressures in the patient by way of a thermal filament located on the catheter. The second physiological sensor can additionally or alternatively measure cardiac output using a bolus thermodilution method.

The adaptive calibration can be based on a time-varying linear scaling and an offset calculated using a least mean-square error solution. Measurement values within the first data can be time averaged over a time window length corresponding to a periodicity of measurements of the second physiological sensor. The time averaged measurement values can be weighted based on a standard deviation of the measurements from each of the first physiological sensor and the second physiological sensor. It can be determined, if a measurement value exceeds a pre-defined standard of deviation value, and the measurement can be characterized as good if it does not exceeds the pre-defined standard of deviation value and the measurement can be characterized as bad if it exceeds the pre-defined standard of deviation value.

The time averaged measurement values can be weighted based on a forgetting factor.

In an interrelated aspect, first data is continuously received that is generated by a first physiological sensor measuring at least one physiological parameter of a patient. In addition, second data is continuously received that is generated by a second physiological sensor concurrently measuring at least one physiological parameter of the patient. The first physiological sensor measures at least one physiological parameter at a higher bandwidth with lower precision as compared to the second physiological sensor. The continuously received first data is adaptively calibrated using the continuously received data to result in a continually updating calibrated measurement. Data characterizing the continually updating calibrated measurement can be provided (e.g., displayed in an electronic visual display, transmitted to a remote computing device, loaded into memory, stored in physical persistence, etc.)

Non-transitory computer program products (i.e., physically embodied computer program products) are also described that store instructions, which when executed by one or more data processors of one or more computing systems, cause at least one data processor to perform operations herein.

Similarly, computer systems are also described that can include one or more data processors and memory coupled to the one or more data processors. The memory can temporarily or permanently store instructions that cause at least one processor to perform one or more of the operations described herein. Such systems can include one or more of the first physiological sensor and the second physiological sensor.

In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g., the Internet, a wireless wide area network, a local area network, a wide area network, a wired network, or the like), via a direct connection between one or more of the multiple computing systems, etc.

The subject matter described herein provides many technical advantages. For example, the current subject matter can provide enhanced physiological measurements by calibrating the output of a first physiological sensor having a greater bandwidth with lower precision using the output of a second physiological sensor having a lower bandwidth with higher precision. In particular, in some implementations, the current subject matter provides enhanced APCO measurements that provide accuracy similar to CCO or ICO measurements while also tracking cardiac output variations that can only be detected using a higher bandwidth.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a logical diagram illustrating a hemodynamic monitor in communication with two physiological sensors affixed to a patient;

FIG. 2 is a simulated measurements diagram illustrating arterial pressure cardiac output, continuous cardiac output, adaptively and linearly calibrated arterial cardiac output, and true cardiac output in relation to one another;

FIG. 3A is a first diagram of real measurements illustrating arterial pressure cardiac output, continuous cardiac output, and adaptively and linearly calibrated arterial cardiac output, in relation to one another;

FIG. 3B is a second diagram of real measurements illustrating arterial pressure cardiac output, continuous cardiac output, and adaptively and linearly calibrated arterial cardiac output, in relation to one another;

FIG. 4 is a diagram illustrating calibration of a hemodynamic measurement by a hemodynamic monitor such as in FIG. 1; and

FIG. 5 is a diagram illustrating a computing device for implementing aspects of the current subject matter.

### DETAILED DESCRIPTION

The current subject matter is directed to systems, methods, and articles that use a lower bandwidth physiological sensor measurement (i.e., a measurement that is taken less frequently) to calibrate a higher bandwidth physiological measurement (i.e., a measurement that is take more frequently than the lower bandwidth physiological sensor measurement) to provide more precise characterization of the condition of a patient (which in turn results in improved patient care). While the current subject matter provides examples for the calculation of cardiac output (CO), unless otherwise specified, the current subject matter can be applicable to other types of physiological sensors in which different underlying measurement techniques are utilized to measure a same or similar physiological condition and one such technique has lower bandwidth/resolution as compared to one or more other techniques. Such measurements can relate to both hemodynamic as well as non-hemodynamic physiological measurements.

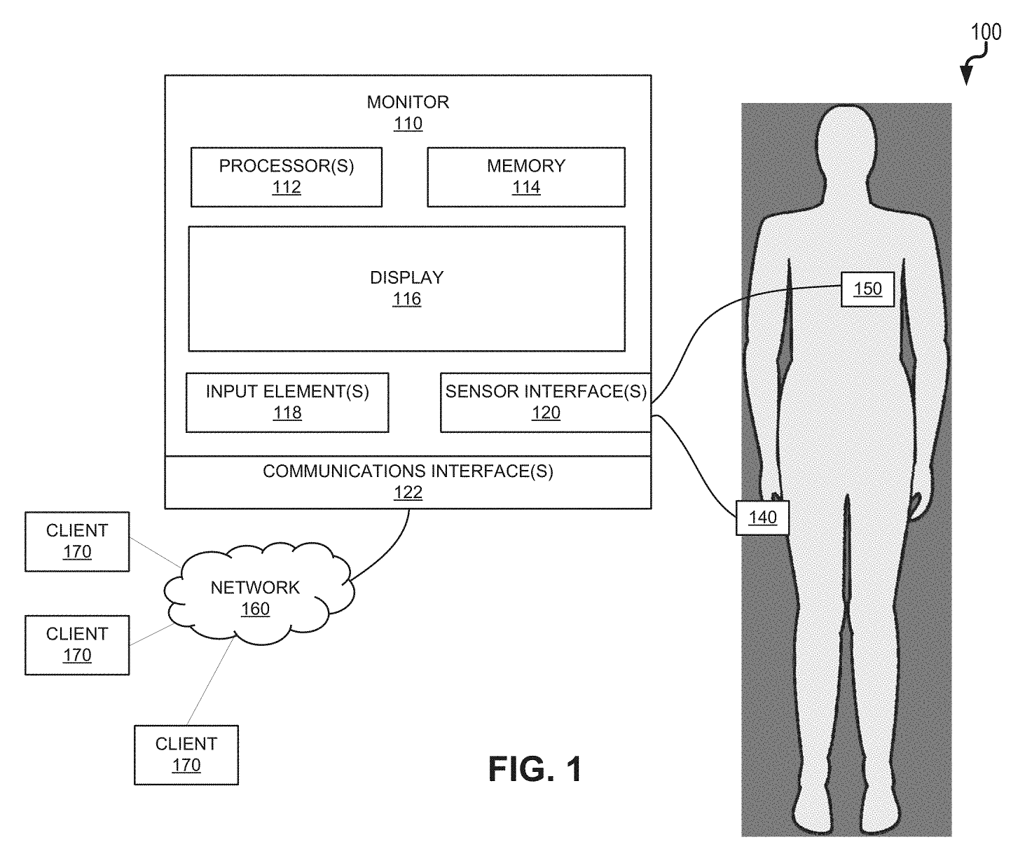

FIG. 1 is a diagram 100 in which a hemodynamic (HD) monitor 110 is configured to receive data characterizing various measured physiological parameters of a patient 130 from a first physiological sensor 140 and a second physiological sensor 150. The HD monitor 110 includes at least one programmable data processor 112 (which may have multiple processing cores), memory 114 for storing instructions for execution by the at least one programmable processor 112, an electronic visual display 116 for rendering a graphical user interface for displaying information that characterizes the measured physiological parameters. Such information can take various forms including waveforms, numerical indications, categorical indications, and the like. The HD monitor 110 can also include various interface input elements 118 which can be physically manipulated to affect operation of the HD monitor 110 such as what information is being displayed on the display 116 and/or configuration information for the first physiological sensor 140 and/or the second physiological sensor 150. In addition or in the alternative, the display 116 can comprise a touch-screen interface allowing users to select graphical user interface elements directly.

The HD monitor 110 can further include a sensor interface 120 that enables data to be received from and optionally additionally transmitted to one or more physiological sensors including the first physiological sensor 140 and the second physiological sensor 150. The sensor interface 120 can communicate with the first physiological sensor 140 and/or the second physiological sensor 150 using a physical wired connection and/or using a wireless data protocol. The HD monitor 110 can also include at least one communications interface 122 that can enable direct or indirect communication with one or more remote client computing systems 170 via a wired and/or wireless network 160. For example, the HD monitor 110 can convey/exchange data with a remote computing system (e.g., the HD monitor 110 can transmit the physiological measurements for storage by a remote database/cloud-storage service, the HD monitor 110 can receive contextual/historical information about the patient which can be used herein, etc.).

The first physiological sensor 140 can be or include a peripheral artery pressure sensor or one or more finger cuffs that can be used to calculate various hemodynamic parameters including stroke volume, stroke volume variation, APCO, systemic vascular resistance (SVR) and continuous blood pressure (cBP). The finger cuffs can perform real-time finger pressure measurements using a volume clamp method at a sampling rate of, for example, 1000 times per second.

The second physiological sensor 150 can be or include a pulmonary artery catheter (PAC) such as a Swan-Ganz catheter. Such catheter can be inserted into a pulmonary artery of the patient 130 to detect direct, simultaneous measurement of pressures in the right atrium, right ventricle, pulmonary artery, and the filling pressure of the left atrium of the patient 130 by way of a thermal filament located on the catheter and using thermodilution principles. In addition or in the alternative, the second physiological sensor 150 can be used to measure CO using bolus thermodilution methods. The second physiological sensor 150 can be used to implement the CCO or ICO algorithms.

The techniques described herein can be processed by the at least one programmable data processor 112 of the HD monitor 110 and/or such processing many be offloaded to one or more of the first physiological sensor 140 or the second physiological sensor 150 or a remote client computing device 170 (which may access the underlying data via the communications interface(s) 122).

With the current arrangement, the CCO or ICO algorithm as provided by the second physiological sensor 150 can act as a calibration for a relatively high bandwidth APCO algorithm as provide by the first physiological sensor 140 in order to estimate the patient's 130 time varying cardiac output, c(t), with accuracy akin to a CCO/ICO algorithm and with bandwidth akin to an APCO algorithm. While the following refers to the second physiological sensor 150 as implementing the CCO algorithm, the thermal dilution input measurements may come from a CCO, an ICO, or a mix of ICO or CCO algorithms. In addition, while the following only refers to CO algorithm output parameter, the current calibration techniques can be applied to other hemodynamic parameter outputs from both algorithms.

One approach employed by the HD monitor 110 can iteratively compute a time-varying linear scaling, A[n], and an offset, B[n], that update an APCO measurement generated by the first physiological sensors 150, m_{F}[n], as follows

\hat{m}[n]=A[n]m_{F}[n]+B[n] (1)

Parameters A[n] and B[n] can be functions of the past history of CO estimates. These functions can act to adjust the APCO measurement to more closely correlate with a CCO measurement. As used herein m_{F} can be referred to as “measurement fast”, but relatively inaccurate measurement and m_{A} can refer to as “measurement accurate”, but relatively low bandwidth.

Namely, A[n] and B[n] can be a least mean-square error solution to the following linear equations that arise by replacing \hat{m}[n] in eq. 1 with CCO measurements and m_{F}[n] with averaged APCO measurements,

$$[ \begin{matrix}{m}_{A} [ 0 ] \\ {m}_{A} [ 1 ] \\ ⋮ \\ {m}_{A} [ n ]\end{matrix} ] = [ \begin{matrix}{\overline{m}}_{F} [ 0 ] & 1 \\ {\overline{m}}_{F} [ 1 ] & 1 \\ ⋮ & \\ {\overline{m}}_{F} [ n ] & 1\end{matrix} ] [ \begin{matrix}A [ n ] \\ B [ n ]\end{matrix} ] \\ {m}_{A} [ n ] = [ \begin{matrix}{\overline{m}}_{F} [ n ] & 1\end{matrix} ] [ \begin{matrix}A [ n ] \\ B [ n ]\end{matrix} ] \qquad ( 2 )$$

where

m_{A}[n]=[m_{A}[0],m_{A}[1], . . . ,m_{A}[n−1],m_{A}[n]]^{T} (3)

is a sequence of discrete CCO estimates arriving at times,

t_{A}[0],t_{A}[1], . . . ,t_{A}[n−1],t_{A}[n] (4)

and where each time value, t_{A}[n], represents the average value of c(t) over an immediately preceding window of time, w A[n], given by the corresponding sequence,

w_{A}[0],w_{A}[1], . . . ,w_{A}[n−1],w_{A}[n] (5)

The sequence of APCO estimates,

m̅_{F}[n]=[m̅_{F}[n],m̅_{F}[n−1], . . . ,m̅_{F}[1],m̅_{F}[0]]^{T} (6)

can represent averages over a time period that is as close as possible to the averaging time window corresponding to ma[n]. An APCO measurement with a line over it, m̅_{F}[k], signifies the averaging time adjustment for m_{F}[k].

An optimum least squares solution for [A[n]B[n]]^{T} can minimize the weighted squared error,

$${min}_{A , B} {e}^{2} = {min}_{A , B} {‖ {m}_{A} - [ {\overline{m}}_{F} 1 ] [ \begin{matrix}A \\ B\end{matrix} ] ‖}_{Ω}^{2} = {min}_{A , B} [ {m}_{A}^{T} - [ A B ] [ \begin{matrix}{\overline{m}}_{F}^{T} \\ {1}^{T}\end{matrix} ] ] Ω [ {m}_{A} - [ {\overline{m}}_{F} 1 ] [ \begin{matrix}A \\ B\end{matrix} ] ] \qquad ( 7 )$$

where for clarity, the dependency on n is not shown. The positive definite matrix,

$$Ω = [ \begin{matrix}ω [ 0 ] & 0 & … & 0 \\ 0 & ω [ 1 ] & ⋱ & ⋮ \\ ⋮ & ⋱ & ⋱ & 0 \\ 0 & … & 0 & ω [ n ]\end{matrix} ] \qquad ( 8 )$$

weights by ω[n] the relative information of the n^{th} measurement. Measurements that are known to be more accurate provide more information and could be weighted higher, while those that are less accurate provide less information and could be weighted lower. For example, if one had the standard deviations for each accurate and averaged fast measurement, σ_{A}[n]+σ_{F}[n], a reasonable choice for each weight could be

$$ω [ n ] = \frac{1}{{σ}_{A} [ n ] + {σ}_{F} [ n ]} \qquad ( 9 )$$

However, if one could classify which measurements were “good” and which were “bad”, another option for the weighting parameters could be

$$ω [ n ] = \{ \begin{matrix}1 & if measurement is good \\ 0 & if measurement is bad\end{matrix} \qquad ( 10 )$$

The important point is that any measure of relative measurement information could be used for the weighting. Weighting can be ignored by setting all weights the same, e.g. ω[n]=1.

For computing the average, the HD monitor 110 can, for example, save current and past values of the APCO measurements,

m_{F}[k]=[m_{F}[k],m_{F}[k−1], . . . ,m_{F}[k−N+1]]^{T} (11)

with corresponding sample times,

t_{F}[k]=[t_{F}[k],t_{F}[k−1],t_{F}[k−2], . . . ,t_{F}[k−N+1]]^{T} (12)

and with corresponding averaging window lengths, w_{F}[k],

w_{F}[k]=[w_{F}[k],w_{F}[k−1],w_{F}[k−2], . . . ,w_{F}[k−N+1]]^{T} (13)

Measurement m_{F}[k] corresponds to time t_{F}[k]−w_{F}[k]/2. N is sufficiently large to cover the worst case delay between a CCO measurement and an APCO measurement ω, plus the worst case CCO averaging time window, _{A}[n]. Generally, the averaging window lengths for an APCO algorithm are constant.

The discrete time index, k, for APCO estimates is different from index n for CCO estimates, because in general APCO and CCO algorithm estimates are (a) not synchronized to a common sampling clock, (b) do not have the same sample time intervals, and (c) nor do they have simple integer or rational fraction related sample time intervals.

Multiplying both sides of eq. 2 by Ω and transposing the right hand side matrix results in the following equivalent linear equations,

$$[ \begin{matrix}{S}_{FA} [ n ] \\ {S}_{A} [ n ]\end{matrix} ] = [ \begin{matrix}{S}_{FF} [ n ] & {S}_{F} [ n ] \\ {S}_{F} [ n ] & M [ n ]\end{matrix} ] [ \begin{matrix}A [ n ] \\ B [ n ]\end{matrix} ] \qquad ( 14 )$$

where

$$\begin{matrix}{S}_{F} [ n ] = {\sum }_{k = 0}^{n} {ω}^{2} [ k ] {\overline{m}}_{F} [ k ] & ( 15 ) \\ {S}_{FF} [ n ] = {\sum }_{k = 0}^{n} {ω}^{2} [ k ] {\overline{m}}_{F}^{2} [ k ] & ( 16 ) \\ {S}_{FA} [ n ] = {\sum }_{k = 0}^{n} {ω}^{2} [ k ] {\overline{m}}_{F} [ k ] {m}_{A} [ k ] & ( 17 ) \\ {S}_{A} [ n ] = {\sum }_{k = 0}^{n} {ω}^{2} [ k ] {m}_{A} [ k ] & ( 18 ) \\ M [ n ] = {\sum }_{k = 0}^{n} {ω}^{2} [ k ] & ( 19 )\end{matrix}$$

All key parameters used to solve for (adapt) A[n] and B[n] can be updated iteratively for new CCO, m_{A}[n+1], and averaged APCO, m_{F}[n+1], measurements as follows,

S_{F}[n+1]=S_{F}[n]+ω²[n+1]m̅_{F}|[n+1] (20)

S_{FF}[n+1]=S_{FF}[n]+ω²[n+1]m̅_{F}²[n+1] (21)

S_{FA}[n+1]=S_{FA}[n]+ω²[n+1]m̅_{F}[n+1]m_{A}[n+1] (22)

S_{A}[n+1]=S_{A}[n]+ω²[n+1]m_{A}[n+1] (23)

M[n+1]=M[n]+ω²[n+1] (24)

However, a problem with these iterations is that measurements far in the past have the same contribution to the solution as more recent measurements. In fact, averaging that includes increasingly larger numbers of measurements, implies that more recent measurements will have less and less overall impact on the calibration over time.

One fix to this problem discounts past measurements using a “forgetting parameter”, 0<γ<1,

S_{F}[n+1]=γS_{F}[n]+ω²[n+1]m̅_{F}[n+1] (25)

S_{FF}[n+1]=γS_{FF}[n]+ω²[n+1]m̅_{F}²[n+1] (26)

S_{FA}[n+1]=γS_{FA}[n]+ω²[n+1]m̅_{F}[n+1]m_{A}[n+1] (27)

S_{A}[n+1]=γS_{A}[n]+ω²[n+1]m_{A}[n+1] (28)

M[n+1]=γM[n]+ω²[n+1] (29)

Setting γ to a small value or “high forgetting” places more weight on the most recent measurement relative to the past, while setting it to a value near 1 or “low forgetting” places more weight on the past.

A second fix can use a constant number P of the most recent measurements by iteratively subtracting the oldest measurement from the totals,

S_{F}[n+1]=S_{F}[n]+ω²[n+1]m̅_{F}[n+1]−ω²[n+1−P]m̅_{F}[n+1−P] (30)

S_{FF}[n+1]=S_{FF}[n]+ω²[n+1]m̅_{F}²[n+1]−ω²[n+1−P]m̅_{F}²[n+1−P] (31)

S_{FA}[n+1]=S_{FA}[n]+ω²[n+1]m̅_{F}[n+1]m_{A}[n+1]−ω²[n+1−P]m̅_{F}[n+1−P]m_{A}[n+1−P] (32)

S_{A}[n+1]=S_{A}[n]+ω²[n+1]m_{A}[n+1]−ω²[n+1−P]m_{A}[n+1−P] (33)

M[n+1]=M[n]+ω²[n+1]−ω²[n+1−P] (34)

In one possible variation to solve linear eq. 14, a solution can be determined by way of inverting the matrix. In particular, inverting the right hand side matrix of eq. 14 yields the following solution for A[n] and B[n],

$$[ \begin{matrix}A [ n ] \\ B [ n ]\end{matrix} ] = \frac{[ \begin{matrix}M [ n ] & - {S}_{F} [ n ] \\ - {S}_{F} [ n ] & {S}_{FF} [ n ]\end{matrix} ]}{D [ n ]} [ \begin{matrix}{S}_{FA} [ n ] \\ {S}_{A} [ n ]\end{matrix} ] \\ [ \begin{matrix}A [ n ] \\ B [ n ]\end{matrix} ] = [ \begin{matrix}( M [ n ] {S}_{FA} [ n ] - {S}_{F} [ n ] {S}_{A} [ n ] ) / D [ n ] \\ - {S}_{F} [ n ] {S}_{FA} [ n ] + {S}_{FF} [ n ] {S}_{A} [ n ] ) / D [ n ]\end{matrix} ] \qquad ( 35 )$$

where determinant

D[n]=M[n]S_{FF}[n]−(S_{F}[n])² (36)

provides a means to assess numerical conditioning. The calibration parameters should not be updated if D[n] is close to 0.

Whether a finite, equally weighted measurement history is utilized by the HD monitor 110, or a forgetting factor iteratively weighted infinite past is utilized, linear eq. 14 and its solution eq. 35 are exactly the same. Only the iterative updates for time varying equation parameters change depending on which iterative update method is being used.

Other linear equation solution methods that can be utilized by the HD monitor 110 include, without limitation, Gaussian elimination, QR factorization, Householder reflectors, and the like. Solution via multiplication by orthogonal matrices (“Householder reflectors”), for example, is a numerically stable method that does not amplify errors.

In practice, it is expected that to have lower likelihood for greater differences of scaling parameter A from 1 or for greater differences of offset parameter B from 0. Adding a “regularization” term to the weighted least squares optimization problem of eq. 7 provides a new optimization problem that takes into account the lower likelihood of large A−1 or B,

$${min}_{A , B} {e}^{2} {min}_{A , B} \{ {‖ {m}_{A} - [ {\overline{m}}_{F} 1 ] [ \begin{matrix}A \\ B\end{matrix} ] ‖}_{Ω}^{2} + {α ( A - 1 )}^{2} + β {B}^{2} \} \qquad ( 37 )$$

where for clarity, the dependency on n is not shown. The first term is the same least squares fit as eq. 7. The second term is a parameter constraint that penalizes scaling parameter A being different from 1, where the constant α>0 is a relative weight on the “importance” of this constraint relative to the other terms. The third term is parameter constraint that penalizes the offset parameter B being different from 0. The constant β>0 is a relative weight on the importance of this constraint relative to the other terms. For example, if keeping B close to 0 has almost no importance, while keeping A close to 1 has relatively high importance, α=0.9, β=0.1 weights the latter 9 times higher than the former. The first term always has a weighting of 1.0, making it more important in this case than the parameter constraints. If α=β=0, this becomes equivalent to the original eq. 7 least squares optimization.

Equating the derivative of eq. 37 to zero with respect to A and B, assuming either a finite or a forgetting factor weighted measurement history, yields the following linear equations for the regularized least squares optimization,

$$[ \begin{matrix}{S}_{FA} [ n ] + α \\ {S}_{A} [ n ]\end{matrix} ] = [ \begin{matrix}{S}_{FF} [ n ] + α & {S}_{F} [ n ] \\ {S}_{F} [ n ] & M [ n ] + β\end{matrix} ] [ \begin{matrix}A [ n ] \\ B [ n ]\end{matrix} ] \qquad ( 38 )$$

with the following matrix inverse solution,

$$[ \begin{matrix}A [ n ] \\ B [ n ]\end{matrix} ] = \frac{[ \begin{matrix}M [ n ] + β & - {S}_{F} [ n ] \\ - {S}_{F} [ n ] & {S}_{FF} [ n ] + α\end{matrix} ]}{D [ n ]} [ \begin{matrix}{S}_{FA} [ n ] + α \\ {S}_{A} [ n ]\end{matrix} ] \\ D [ n ] = ( M [ n ] + β ) ( {S}_{FF} [ n ] + α ) - {( {S}_{F} [ n ] )}^{2} \\ [ \begin{matrix}A [ n ] \\ B [ n ]\end{matrix} ] = [ \begin{matrix}( {S}_{FA} [ n ] + α ) ( M [ n ] + β ) - {S}_{F} [ n ] {S}_{A} [ n ] ) / D [ n ] \\ - {S}_{F} [ n ] ( {S}_{FA} [ n ] + α ) + ( {S}_{FF} [ n ] + α ) {S}_{A} [ n ] ) / D [ n ]\end{matrix} ] \qquad ( 39 )$$

The HD monitor 110 can implement the techniques provided herein using a variety of software and/or hardware implementations. In one implementation, a Python module can include a measurement “Combiner class” that implements the adaptive least squares mathematics to combine a fast, but less accurate algorithm measurement with a second algorithm measurement that is slower, but relatively more accurate. The implementation provides an option to use a forgetting factor γ, or to use a finite number of past accurate algorithm estimates. In the latter case, the number of past measurements is a function of γ, namely P=1/(1−γ). Variable names and numerical implementation mnemonically match mathematical notation in this document.

The Combiner class can assume that different fast or accurate measurements may arrive asynchronously at different times. There can be separate interface functions to update the algorithm with these measurements. A first function (e.g., function UpdateFast(c,t,w)) can provide the means to inform the algorithm of a new fast measurement m along with the measurement time, t, and the averaging time window, w. The measurement represents time window [t−w,t]. A second function (e.g., UpdateAccurate (c,t,w)) is an analogous function to inform the algorithm of a new accurate algorithm measurement. A third function (e.g., function Combine( ) can return a combined measurement.

If measurement algorithm averaging is uniform or symmetrical over window w_{A}[k] for measurement m[k], the measurement corresponds to the time point, t_{A}[k]−w_{A}[k]/2. If averaging is not uniform or symmetrical, the measurement algorithm implementation should provide an input/output Delay( ) function to return how far in the past that the current measurement corresponds. In general, it is typically better to use a measurement algorithm Delay( ) function regardless, in order to keep the measurement combining algorithm independent of the individual fast or accurate measurement algorithm averaging policies.

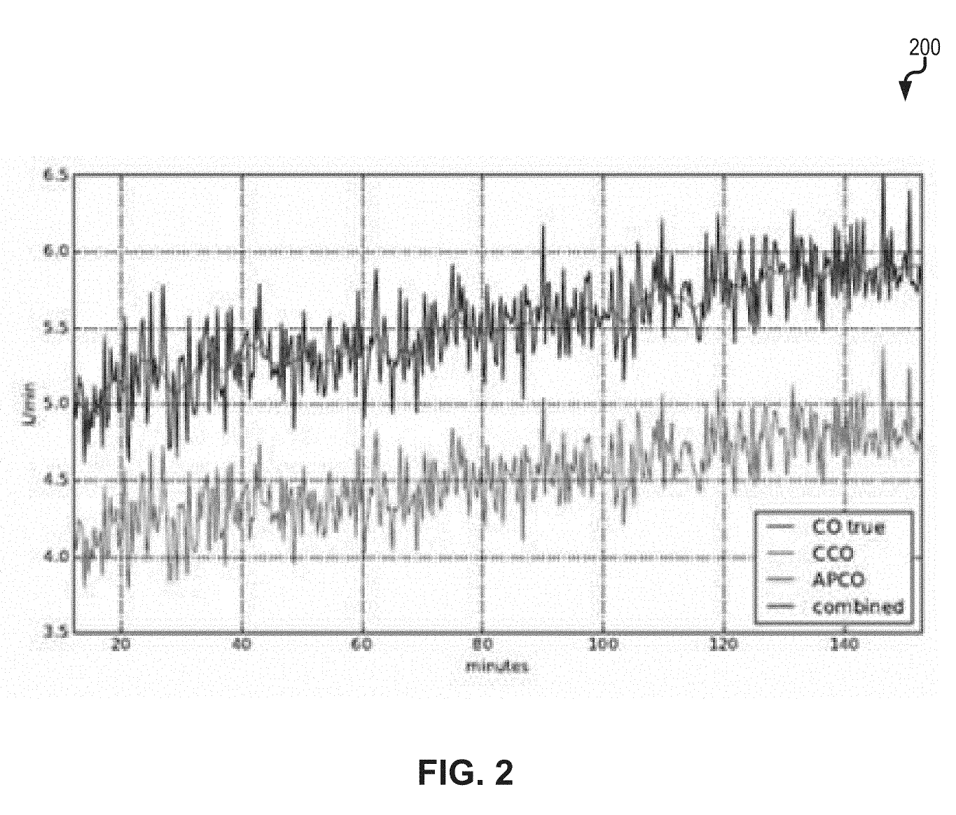

In one simulation, a random “true CO” was generated every 20 seconds, from which the CCO measurements were an average of this sequence over 3 minutes. To simulate less accuracy, the APCO measurements were scaled and an offset adjustment of these measurements was applied. Diagram 200 of FIG. 2 shows a simulation run and Table 1 lists a mean-square error for each of the measurements for several simulation runs. FIG. 2 shows that the combined CO estimate follows both the low bandwidth trend of the CCO measurement and the higher bandwidth variation of the APCO estimate. Table 1 shows that the combined estimate mean-square error is almost an order of magnitude better than the CCO (accurate, but slow algorithm) estimate. In FIG. 2, a first line pattern is a simulated “true CO”, a second line pattern is a simulated accurate but averaged CCO, and a third line is an un-averaged, but less accurate APCO, with the fourth line being a combined measurement. The simulation used a finite history P=10 (minutes) of CCO results. Plots can be shifted to account for algorithm input/output delay to align corresponding measurements. Measurement information weighting values w[n] were random between 0 and 1.

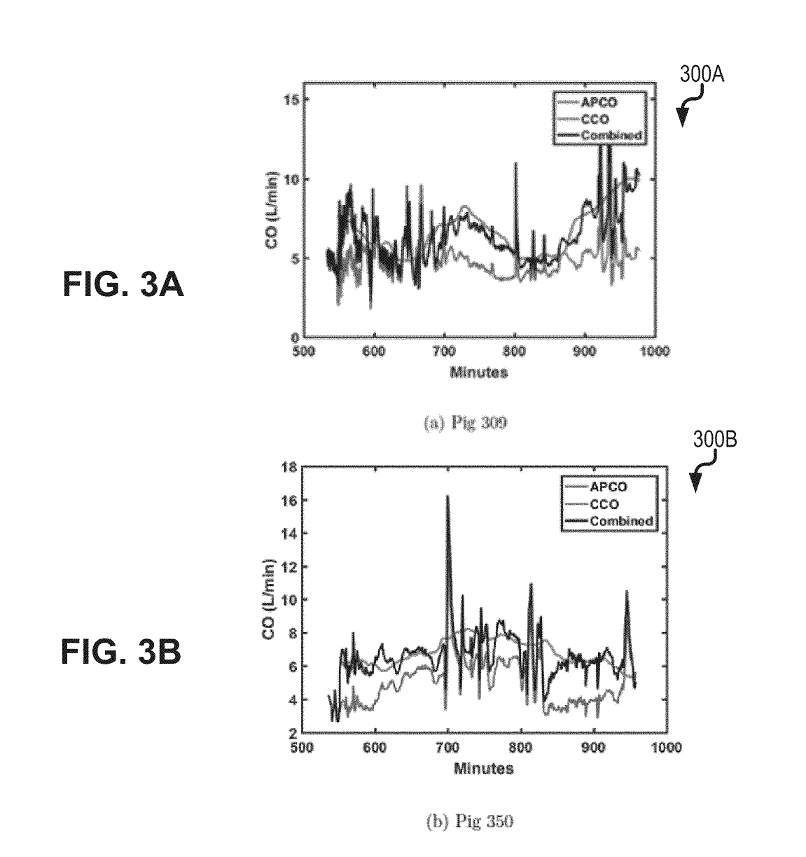

FIGS. 3A and 3B are diagrams 300A, 300B that illustrate operation on challenging porcine temperature and pressure waveforms where vasopressor administration along with induced bleeding, and fluid administration caused intentional significant swings in CO not common in human patients. As can be seen, the combined measurement has similar average (accuracy) of the CCO algorithm along with variable CO swings highly correlated to the swings of the APCO algorithm.

Table 1 below shows a comparison of the mean-square CO estimation errors (L/min) for the three algorithms (APCO, CCO and the combined techniques provided here) using 9600 measurement samples per simulation run. The combined technique used a finite history P=10 (minutes) of CCO results to calibrate APCO.

TABLE 1

| Run# | APCO | CCO | Combined |
|---|---|---|---|
| 1 | 1.004 | 0.036 | 0.005 |
| 2 | 1.003 | 0.036 | 0.005 |
| 3 | 1.004 | 0.036 | 0.005 |
| 4 | 1.002 | 0.036 | 0.005 |
| 5 | 1.002 | 0.036 | 0.005 |

FIG. 3 are diagrams 300A and 300 B in which a first line pattern is FloTrac APCO, a second line pattern is a CCO measurement, and a third line pattern is the combined measurement. With forgetting factor γ=0.8, the approximate finite history to fit P=5 minutes. Regularization parameters α=1.0 and β=0.5. There was no variable measurement information weighting (w[n]=1).

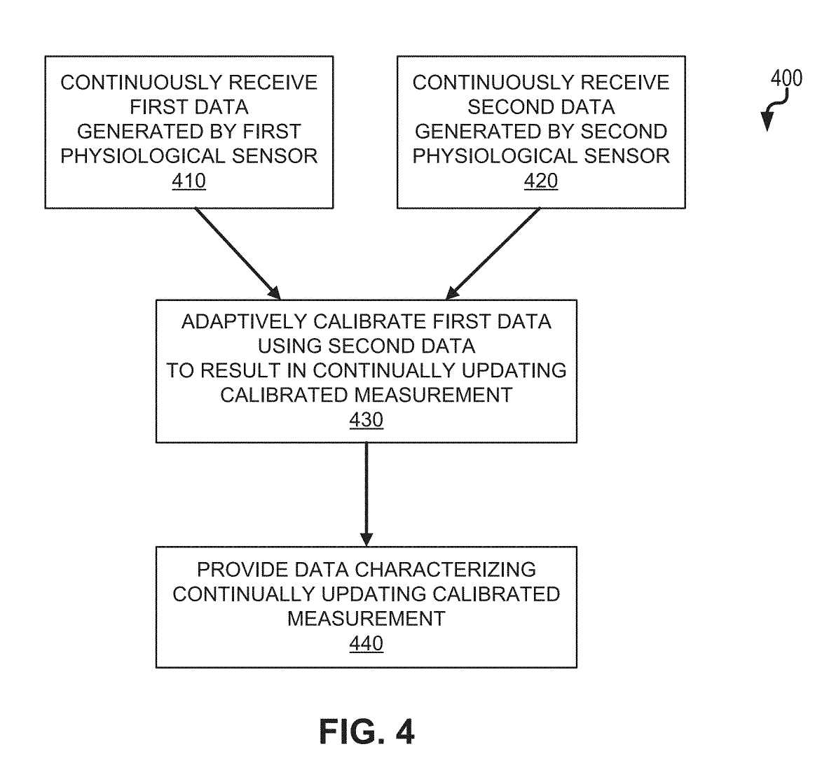

FIG. 4 is a diagram 400 in which, at 410, first data that is generated by a first physiological sensor measuring at least one hemodynamic parameter of a patient is continuously received. In addition, at 420, second data that is generated by a second physiological sensor concurrently measuring the at least one hemodynamic parameter of the patient is continuously received. The first physiological sensor measures the at least one hemodynamic parameter at a higher bandwidth with lower precision as compared to the second physiological sensor. The continuously received first data is adaptively calibrated, at 430, using the continuously received second data to result in a continually updating calibrated measurement. Data characterizing the calibrated measurement is, at 440, provided (e.g., displayed in an electronic visual display, loaded into memory, stored in physical data persistence, and/or transmitted to a remote computing device, etc.).

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system can include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, can include machine instructions for a programmable processor, and/or can be implemented in a high-level procedural language, an object-oriented programming language, a functional programming language, a logical programming language, and/or in assembly/machine language. As used herein, the term “computer-readable medium” refers to any computer program product, apparatus and/or device, such as for example magnetic discs, optical disks, solid-state storage devices, memory, and Programmable Logic Devices (PLDs), used to provide machine instructions and/or data to a programmable data processor, including a machine-readable medium that receives machine instructions as a computer-readable signal. The term “computer-readable signal” refers to any signal used to provide machine instructions and/or data to a programmable data processor. The computer-readable medium can store such machine instructions non-transitorily, such as for example as would a non-transient solid-state memory or a magnetic hard drive or any equivalent storage medium. The computer-readable medium can alternatively or additionally store such machine instructions in a transient manner, such as for example as would a processor cache or other random access memory associated with one or more physical processor cores.

The computer components, software modules, functions, data stores and data structures described herein can be connected directly or indirectly to each other in order to allow the flow of data needed for their operations. It is also noted that a module or processor includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality can be located on a single computer or distributed across multiple computers depending upon the situation at hand.

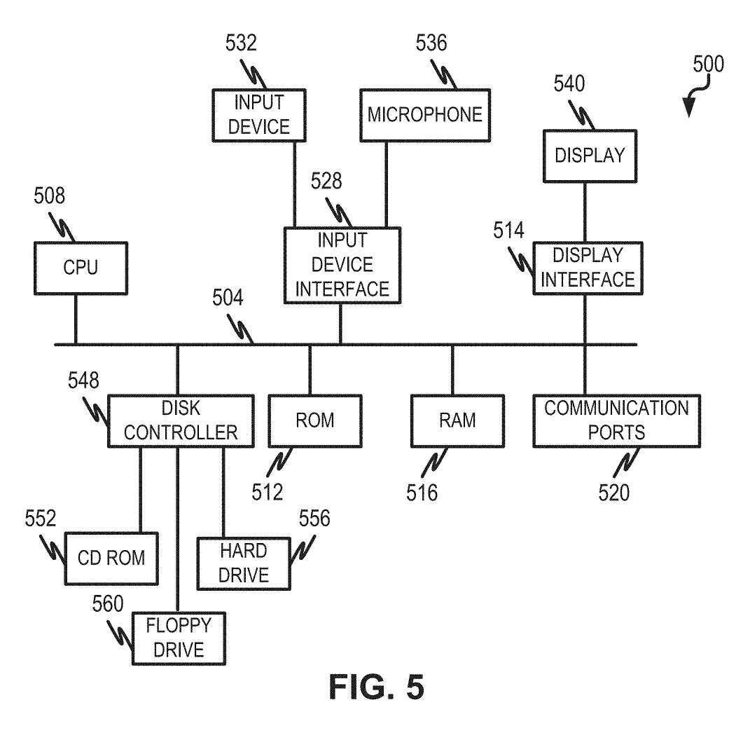

FIG. 5 is a diagram 500 illustrating a sample computing device architecture for implementing various aspects described herein. A bus 504 can serve as the information highway interconnecting the other illustrated components of the hardware. A processing system 508 labeled CPU (central processing unit) (e.g., one or more computer processors/data processors at a given computer or at multiple computers), can perform calculations and logic operations required to execute a program. A non-transitory processor-readable storage medium, such as read only memory (ROM) 512 and random access memory (RAM) 416, can be in communication with the processing system 508 and can include one or more programming instructions for the operations specified here. Optionally, program instructions can be stored on a non-transitory computer-readable storage medium such as a magnetic disk, optical disk, recordable memory device, flash memory, solid-state or other physical storage medium.

In one example, a disk controller 548 can interface one or more optional disk drives to the system bus 504. These disk drives can be external or internal floppy disk drives such as 560, external or internal CD-ROM, CD-R, CD-RW or DVD, or solid state drives such as 552, or external or internal hard drives 556. As indicated previously, these various disk drives 552, 556, 560 and disk controllers are optional devices. The system bus 504 can also include at least one communication port 520 to allow for communication with external devices either physically connected to the computing system or available externally through a wired or wireless network. In some cases, the communication port 520 includes or otherwise comprises a network interface.

To provide for interaction with a user, the subject matter described herein can be implemented on a computing device having a display device 540 (e.g., a CRT (cathode ray tube) or LCD (liquid crystal display) monitor, touchscreen, etc.) for displaying information obtained from the bus 504 to the user and an input device 532 such as keyboard and/or a pointing device (e.g., a mouse or a trackball) and/or a touchscreen by which the user can provide input to the computer. Other kinds of input devices 532 can be used to provide for interaction with a user as well; for example, feedback provided to the user can be any form of sensory feedback (e.g., visual feedback, auditory feedback by way of a microphone 536, or tactile feedback); and input from the user can be received in any form, including acoustic, speech, or tactile input. In the input device 532 and the microphone 536 can be coupled to and convey information via the bus 504 by way of an input device interface 528. Other computing devices, such as dedicated servers, can omit one or more of the display 540 and display interface 524, the input device 532, the microphone 536, and input device interface 528.

In the descriptions above and in the claims, phrases such as “at least one of” or “one or more of” can occur followed by a conjunctive list of elements or features. The term “and/or” can also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A method for implementation by one or more programmable data processors forming part of at least one computing device, the method comprising:
continuously receiving first data generated by a first physiological sensor measuring at least one hemodynamic parameter of a patient;
continuously receiving second data generated by a second physiological sensor concurrently measuring the at least one hemodynamic parameter of the patient, the first physiological sensor measuring the at least one hemodynamic parameter at a higher bandwidth with lower precision as compared to the second physiological sensor;
adaptively calibrating the continuously received first data using the continuously received data to result in a continually updating calibrated measurement; and
providing data characterizing the continually updating calibrated measurement.

2. The method of claim 1, wherein the providing data comprises one or more of: displaying the data characterizing the calibrated measurement in an electronic visual display, transmitting the data characterizing the calibrated measurement to a remote computing system, loading the data characterizing the calibrated measurement into memory, or storing the data characterizing the calibrated measurement in physical data persistence.

3. The method of claim 1, wherein the at least one hemodynamic parameter is cardiac output.

4. The method of claim 1, wherein the first physiological sensor is used to measure arterial pressure cardiac output.

5. The method of claim 4, wherein the first physiological sensor comprises a cuff to be placed on an extremity of the patient and utilizing a volume clamp method to calculate at least one hemodynamic parameter selected from a group consisting of: stroke volume, stroke volume variation, APCO, systemic vascular resistance (SVR), or continuous blood pressure (cBP).

6. The method of claim 1, wherein the second physiological sensor is used to measure continuous cardiac output and/or injectate cardiac output.

7. The method of claim 6, wherein the second physiological sensor comprises a pulmonary artery catheter (PAC) that is inserted into a pulmonary artery of the patient to detect cardiac pressures in the patient by way of a thermal filament located on the catheter.

8. The method of claim 6, wherein the second physiological sensor measures cardiac output using a bolus thermodilution method.

9. The method of claim 1, wherein the adaptive calibration is based on a time-varying linear scaling and an offset calculated using a least mean-square error solution.

10. The method of claim 9 further comprising: time averaging measurement values within the first data over a time window length corresponding to a periodicity of measurements of the second physiological sensor.

11. The method of claim 10 further comprising:
weighting the time averaged measurement values based on a standard deviation of the measurements from each of the first physiological sensor and the second physiological sensor.

12. The method of claim 11 further comprising:
determining if a measurement value exceeds a pre-defined standard of deviation value; and
characterizing the measurement value as being a good measurement if it does not exceeds the pre-defined standard of deviation value; or
characterizing the measurement value as being a bad measurement if it exceeds the pre-defined standard of deviation value.

13. The method of 10 further comprising: weighting the time averaged measurement values based on a forgetting factor.

14. A method for implementation by one or more programmable data processors forming part of at least one computing device, the method comprising:
continuously receiving first data generated by a first physiological sensor measuring at least one physiological parameter of a patient;
continuously receiving second data generated by a second physiological sensor concurrently measuring at least one physiological parameter of the patient, the first physiological sensor measuring at least one physiological parameter at a higher bandwidth with lower precision as compared to the second physiological sensor;
adaptively calibrating the continuously received first data using the continuously received data to result in a continually updating calibrated measurement; and
providing data characterizing the continually updating calibrated measurement.

15. A system comprising:
at least one programmable data processor; and
memory storing instructions which, when executed by the at least one programmable data processor, implement operations comprising:
continuously receiving first data generated by a first physiological sensor measuring at least one hemodynamic parameter of a patient;
continuously receiving second data generated by a second physiological sensor concurrently measuring the at least one hemodynamic parameter of the patient, the first physiological sensor measuring the at least one hemodynamic parameter at a higher bandwidth with lower precision as compared to the second physiological sensor;
adaptively calibrating the continuously received first data using the continuously received data to result in a continually updating calibrated measurement; and
providing data characterizing the continually updating calibrated measurement.

16. The system of claim 15 further comprising the first physiological sensor and the second physiological sensor.

17. The system of claim 15, wherein the providing data comprises one or more of: displaying the data characterizing the calibrated measurement in an electronic visual display, transmitting the data characterizing the calibrated measurement to a remote computing system, loading the data characterizing the calibrated measurement into memory, or storing the data characterizing the calibrated measurement in physical data persistence.

18. The system of claim 15, wherein the at least one hemodynamic parameter is cardiac output.

19. The system of claim 15, wherein the first physiological sensor is used to measure arterial pressure cardiac output.

20. The system of claim 19, wherein the first physiological sensor comprises a cuff to be placed on an extremity of the patient and utilizing a volume clamp method to calculate at least one hemodynamic parameter selected from a group consisting of: stroke volume, stroke volume variation, APCO, systemic vascular resistance (SVR), or continuous blood pressure (cBP).

21. The system of claim 15, wherein the second physiological sensor is used to measure continuous cardiac output and/or injectate cardiac output.

22. The system of claim 21, wherein the second physiological sensor comprises a pulmonary artery catheter (PAC) that is inserted into a pulmonary artery of the patient to detect cardiac pressures in the patient by way of a thermal filament located on the catheter.

23. The system of claim 21, wherein the second physiological sensor measures cardiac output using a bolus thermodilution method.

24. The system of claim 15, wherein the adaptive calibration is based on a time-varying linear scaling and an offset calculated using a least mean-square error solution.

25. The system of claim 24, wherein the operations further comprise:
time averaging measurement values within the first data over a time window length corresponding to a periodicity of measurements of the second physiological sensor.

26. The system of claim 24, wherein the operations further comprise:
weighting the time averaged measurement values based on a standard deviation of the measurements from each of the first physiological sensor and the second physiological sensor.

27. The system of claim 26, wherein the operations further comprise:
determining if a measurement value exceeds a pre-defined standard of deviation value; and
characterizing the measurement value as being a good measurement if it does not exceeds the pre-defined standard of deviation value; or
characterizing the measurement value as being a bad measurement if it exceeds the pre-defined standard of deviation value.

28. The system of claim 25, wherein the operations further comprise:
weighting the time averaged measurement values based on a forgetting factor.

29. A system comprising:
at least one programmable data processor; and
memory storing instructions which, when executed by the at least one programmable data processor, implement operations comprising:
continuously receiving first data generated by a first physiological sensor measuring at least one physiological parameter of a patient;
continuously receiving second data generated by a second physiological sensor concurrently measuring at least one physiological parameter of the patient, the first physiological sensor measuring at least one physiological parameter at a higher bandwidth with lower precision as compared to the second physiological sensor;
adaptively calibrating the continuously received first data using the continuously received data to result in a continually updating calibrated measurement; and
providing data characterizing the continually updating calibrated measurement.
